(19) United States
(12) Patent Application Publication
(10) Pub. No.: US 2020/0316130 A1
(43) Pub. Date: Oct. 8, 2020

(54) MATERIALS AND METHODS FOR ENHANCING BONE CELL DIFFERENTIATION

(71) Applicant: Mayo Foundation for Medical Education and Research, Rochester, MN (US)

(72) Inventors: Andre J. van Wijnen, Rochester, MN (US); Amel Dudakovic, Rochester, MN (US)

(21) Appl. No.: 16/326,604

(22) PCT Filed: Aug. 17, 2017
(86) PCT No.: PCT/US2017/047315

§ 371 Date: Feb. 19, 2019

Related U.S. Application Data
(60) Provisional application No. 62/376,841, filed on Aug. 18, 2016.

(51) Int. Cl. A61K 35/28; A61K 35/32; A61K 31/496; A61K 31/403; A61K 47/42

(52) U.S. Cl. CPC A61K 35/28; A61K 35/32; A61K 47/42; A61K 31/403; A61K 31/496

(57) ABSTRACT

This document provides materials and methods for enhancing bone cell differentiation. For example, compositions containing an epigenetic drug, an actin modulator, and/or one or more osteogenic differentiation factors that can be used to enhance bone cell differentiation are provided.

### CROSS-REFERENCE TO RELATED APPLICATIONS

This application claims the benefit of U.S. Patent Application Ser. No. 62/376,841, filed on Aug. 18, 2016. The disclosure of the prior application is considered part of (and is incorporated by reference in) the disclosure of this application.

### BACKGROUND

### 1. Technical Field

This document relates to materials and methods for enhancing bone cell differentiation. For example, this document relates to compositions containing an epigenetic drug, an actin modulator, and/or one or more osteogenic differentiation factors to enhance bone cell differentiation.

### 2. Background Information

Stem cells are undifferentiated biological cells characterized by the ability to differentiate into a broad range of cell types. In the presence of certain differentiation factors, stem cells can be differentiated into specialized cell types.

### SUMMARY

This document provides materials and methods for enhancing bone cell differentiation. For example, this document provides materials and methods for using epigenetic drugs, actin modulators, and/or osteogenic differentiation factors to enhance bone cell differentiation from a stem cell. In some cases, a composition containing an epigenetic drug, an actin modulator, and/or one or more osteogenic differentiation factors can be used to enhance bone cell differentiation.

As demonstrated herein, epigenetic drugs (e.g., that inhibit enhancer of zeste homolog 2 (EZH2)) and actin modulators (e.g., that disrupt actin polymerization) can be used to enhance osteogenic differentiation.

In general, one aspect of this document features a composition comprising, or consisting essentially of, an epigenetic drug, an actin modulator, and one or more osteogenic differentiation factors. The epigenetic drug can inhibit EZH2 polypeptide activity. The epigenetic drug can be GSK126. The actin modulator can inhibit actin polymerization. The actin modulator can be cytochalasin D (CytoD). The one or more osteogenic differentiation factors can be pro-osteogenic differentiation factors. The one or more osteogenic differentiation factors can be selected from the group consisting of Wnt10b, Wnt10a, Wnt6, Pthr1h, DLX5, SP7, and IBSP. The epigenetic drug can be GSK126, the actin modulator can be CytoD, and the one or more osteogenic differentiation factors can be selected from the group consisting of Wnt10b, a sclerostin antibody, BMP2, BMP5, teriparatide, and abaloparatide.

In another aspect, this document features a composition comprising, or consisting essentially of, GSK126, CytoD, and one or more of Wnt10b, a sclerostin antibody, BMP2, BMP5, teriparatide, and abaloparatide.

In another aspect, this document features a method for enhancing bone cell differentiation. The method comprises, or consists essentially of, administering to a stem cell a composition comprising an epigenetic drug, an actin modulator, and one or more osteogenic differentiation factors, wherein the stem cell is differentiated into a bone cell. The stem cell can be an adult stem cell. The adult stem cell can be selected from the group consisting of a mesenchymal stem cell, an adipose-derived stem cell, and a bone marrow-derived stem cell. The adult stem cell can be an adipose-derived stem cell. The bone cell can be an osteocyte. The stem cell can be from a mammal. The mammal can be a human. The epigenetic drug can be GSK126. The actin modulator can be CytoD. The one or more osteogenic differentiation factors can be selected from the group consisting of Wnt10b, a sclerostin antibody, BMP2, BMP5, teriparatide, and abaloparatide. The stem cell can be in vivo. The stem cell can be in vitro. The stem cell can be on a substrate. The substrate can comprise a bone-related extracellular matrix protein. The bone-related extracellular matrix protein can be a collagen.

In another aspect, this document features a method for increasing polypeptide expression in a stem cell. The method comprises, or consists essentially of, contacting the stem cell with a composition comprising an epigenetic drug, an actin modulator, or one or more osteogenic differentiation factors, wherein expression of one or more osteogenic polypeptides is increased. The stem cell can be an adult stem cell. The adult stem cell can be selected from the group consisting of a mesenchymal stem cell, an adipose-derived stem cell, and a bone marrow-derived stem cell. The adult stem cell can be an adipose-derived stem cell. The bone cell can be an osteocyte. The stem cell can be from a mammal. The mammal can be a human. The composition can comprise GSK126. The composition can comprise CytoD. The composition can comprise Wnt10b, a sclerostin antibody, BMP2, BMP5, teriparatide, or abaloparatide. The stem cell can be in vivo. The stem cell can be in vitro.

Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the description and drawings, and from the claims.

### DETAILED DESCRIPTION

This document provides materials and methods for enhancing bone cell differentiation. For example, this document provides materials and methods for using epigenetic drugs, actin modulators, and/or osteogenic differentiation factors to enhance bone cell differentiation from a stem cell (e.g., a human stem cell). In some cases, the materials and methods described herein can be used to enhance differentiation of a stem cell to a bone cell. In some cases, a composition containing an epigenetic drug, an actin modulator, and/or one or more osteogenic differentiation factors can be used to enhance bone cell differentiation.

A composition provided herein (e.g., containing an epigenetic drug, an actin modulator, and one or more osteogenic differentiation factors) can include any appropriate epigenetic drug. An epigenetic drug can activate or inactivate an epigenetic regulator. Examples of epigenetic regulators that can be used to enhance bone cell differentiation include, without limitation, methyltransferases (e.g., N-terminal methyltransferases such as EZH2, histone methyltransferases, and DNA/RNA methyltransferases), histone acetyltransferases (e.g., Gcn5-related N-acetyltransferases), and chromatin-remodelling enzymes. In some cases, an epigenetic regulator can modify DNA or histones within chromatin. In some cases, an epigenetic regulator can inactivate EZH2. An epigenetic regulator that inactivates EZH2 can inhibit EZH2 polypeptide expression or inhibit EZH2 polypeptide activity. Examples of epigenetic drugs that reduce EZH2 polypeptide activity include, without limitation, GSK126, 3-deazaneplanocin A (DZNep), EPZ005687, EI1, UNC1999, and Sinefungin. Examples of epigenetic drugs that reduce EZH2 polypeptide expression include, without limitation, nucleic acid molecules designed to induce RNA interference (e.g., a siRNA molecule or a shRNA molecule), antisense molecules, and miRNAs. EZH2 inhibitors can be readily designed based upon the nucleic acid and/or polypeptide sequences of EZH2. Examples of an EZH2 nucleic acids include, without limitation, the human EZH2 sequence set forth in GenBank® Accession No. U61145 (see, e.g., Version U61145.1, GI No. 1575348). Examples of EZH2 polypeptides include, without limitation, the human EZH2 polypeptide having the amino acid sequence set forth in GenBank® accession Nos: Q15910 (see, e.g., Version Q15910.2, GI No. 3334180), NP_001190178 (see, e.g., Version NP_001190178.1, GI No. 322506101), NP_001190177 (see, e.g., Version NP_001190177.1, GI No. 322506099), NP_001190176 (see, e.g., Version NP_001190176.1, GI No. 322506097), NP_694543 (see, e.g., Version NP_694543.1, GI No. 23510384), and NP_004447 (see, e.g., Version NP_004447.2, GI No. 21361095). For example, GSK126 can be used to inactivate EZH2.

A composition provided herein (e.g., containing an epigenetic drug, an actin modulator, and one or more osteogenic differentiation factors) can include any appropriate actin modulator. An actin modulator can disrupt or enhance actin polymerization. Examples of actin modulators that can be used to enhance bone cell differentiation include, without limitation, CytoD, latrunculin, and phalloidin. In some cases, an actin modulator can disrupt actin polymerization. For example, CytoD can be used to disrupt actin polymerization.

A composition provided herein (e.g., containing an epigenetic drug, an actin modulator, and one or more osteogenic differentiation factors) can include any appropriate osteogenic differentiation factor. An osteogenic differentiation factor can be a pro-osteogenic differentiation factor. For example, an osteogenic differentiation factor can activate one or more of the WNT, BMP, and PTH pathways. In some cases, the one or more osteogenic differentiation factors can include one or more Wnt modulators. In some cases, the one or more osteogenic differentiation factors can include one or more BMPs. In some cases, the one or more osteogenic differentiation factors can include one or more PTH-related molecules. Examples of osteogenic differentiation factors that can be used to enhance bone cell differentiation as described herein include, without limitation, Wnt ligands (e.g., Wnt10b, Wnt10a, and Wnt6), the PTH receptor (Pthr1h), BMP2-responsive genes (e.g., PTH1R, DLX5, SP7, and IBSP), sclerostin antibodies, BMPs (e.g., BMP2 and BMP5), and PTH-related molecules (e.g., teriparatide, and abaloparatide). In some cases, one or more (e.g., two, three, four, five, six, seven, eight, nine, ten, eleven, twelve, or more) osteogenic differentiation factors can be provided to a stem cell to enhance bone cell differentiation.

In some cases, a composition provided herein (e.g., containing an epigenetic drug, an actin modulator, and one or more osteogenic differentiation factors) can include GSK126, CytoD, and one or more of Wnt10b, Pthr1h, PTH1R, DLX5, SP7, and/or IBSP.

In some cases, a composition provided herein (e.g., containing an epigenetic drug, an actin modulator, and one or more osteogenic differentiation factors) can include GSK126, CytoD, and one or more of Wnt10b, a sclerostin antibody, BMP2, BMP5, teriparatide, and/or abaloparatide.

In some cases, a composition containing an epigenetic drug, an actin modulator, and one or more osteogenic differentiation factors can be formulated as a pharmaceutical composition. For example, a composition containing an epigenetic drug, an actin modulator, and one or more osteogenic differentiation factors provided herein can contain a pharmaceutically acceptable carrier for administration to a mammal, including, without limitation, sterile aqueous or non-aqueous solutions, suspensions, and emulsions. Examples of non-aqueous solvents include, without limitation, propylene glycol, polyethylene glycol, vegetable oils, and organic esters. Aqueous carriers include, without limitation, water, alcohol, saline, and buffered solutions. Acceptable carriers also can include physiologically acceptable aqueous vehicles (e.g., physiological saline) or other known carriers for oral administration.

An acceptable aqueous vehicle can be, for example, any liquid solution that is capable of dissolving a composition containing an epigenetic drug, an actin modulator, and one or more osteogenic differentiation factors provided herein and is not toxic to the particular individual receiving the composition. Examples of acceptable aqueous vehicles include, without limitation, saline, water, and acetic acid. Typically, acceptable aqueous vehicles are sterile. An acceptable solid vehicle can be formulated such that compositions containing an epigenetic drug, an actin modulator, and one or more osteogenic differentiation factors provided herein is suitable for oral administration. The dose supplied by each capsule or tablet can vary since an effective amount can be reached by administrating either one or multiple capsules or tablets. Any appropriate pharmaceutically acceptable material such as gelatin and cellulose derivatives can be used as an acceptable solid vehicle. In addition, an acceptable solid vehicle can be a solid carrier including, without limitation, starch, sugar, or bentonite. Further, a tablet or pill formulation of a composition containing an epigenetic drug, an actin modulator, and one or more osteogenic differentiation factors can follow conventional procedures that employ solid carriers, lubricants, and the like. In some cases, a formulation of a composition containing an epigenetic drug, an actin modulator, and one or more osteogenic differentiation factors can be formulated for controlled release.

Any appropriate method can be used to formulate a pharmaceutical composition provided herein (e.g., a pharmaceutical composition containing an epigenetic drug, an actin modulator, and one or more osteogenic differentiation factors). For example, common formulation mixing and preparation techniques can be used to make a composition having the components described herein. In addition, the compositions provided herein can be in any appropriate form. For example, a composition provided herein can be in the form of a solid, liquid, and/or aerosol including, without limitation, powders, crystalline substances, gels, pastes, ointments, salves, creams, solutions, suspensions, partial liquids, sprays, nebulae, mists, atomized vapors, tinctures, pills, capsules, tablets, and gelcaps.

This document also provides methods and materials for using a composition containing an epigenetic drug, an actin modulator, and one or more osteogenic differentiation factors provided herein. In some cases, a composition containing an epigenetic drug, an actin modulator, and/or one or more osteogenic differentiation factors can be used to enhance bone cell differentiation. Any appropriate method can be used to determine whether or not a stem cell has differentiated into a bone cell. For example, osteogenic differentiation can be assessed by increases in alkaline phosphatase activity; increases in mineralization; expression of osteogenic polypeptides runt-related transcription factor 2 (RUNX2), alkaline phosphatase (ALP), osteoprotegerin (OPG) transforming growth factor beta-3 (TGFβ3), transcription factor Sp7 (SP7); and/or decreases in protein levels of epigenetic suppressor of osteogenic differentiation such as EZH2. In some cases, a composition containing an epigenetic drug, an actin modulator, and/or one or more osteogenic differentiation factors can be used to increase expression of one or more osteogenic polypeptides. For example, a composition provided herein can be used to increase expression of RUNX2, ALP, OPG TGFβ3, SP7, or a combination thereof. In humans, a composition containing an epigenetic drug, an actin modulator, and/or one or more osteogenic differentiation factors provided herein can be used to increase expression of a human RUNX2 polypeptide, a human ALP polypeptide, a human OPG polypeptide, a human TGFβ3 polypeptide, a human SP7 polypeptide, or a combination thereof.

A composition containing an epigenetic drug, an actin modulator, and/or one or more osteogenic differentiation factors can be administered to any appropriate stem cell. In some cases, a stem cell that can be differentiated into a bone cell can be an adult stem. Examples of stem cells include, without limitation, mesenchymal stem cell (MSC), adipose-derived stem cell (AMSC), bone marrow-derived stem cell (BMSC), endothelial stem cell, and dental pulp stem cell. A stem cell can be differentiated into any type of bone cell such as osteoclasts, osteoblasts, and osteocytes.

A stem cell can be from any appropriate type of mammal. For example, stem cells from humans and other primates such as monkeys can be differentiated into bone cells as described herein. In some cases, stem cells from dogs, cats, horses, cows, pigs, sheep, rabbits, mice, and rats can be differentiated into bone cells as described herein.

Methods for enhancing bone cell differentiation described herein can include administering a composition provided herein (e.g., a composition containing an epigenetic drug, an actin modulator, and one or more osteogenic differentiation factors) to a stem cell. A composition provided herein can be administered to a stem cell in vivo or in vitro. In some cases, a composition provided herein can be administered to a stem cell in vivo (e.g., administered locally or systemically to a mammal). For example, a composition containing an epigenetic drug, an actin modulator, and one or more osteogenic differentiation factors can be administered systemically by an oral administration to a mammal (e.g., a human). In cases where a composition provided herein is administered to a stem cell in vitro, the composition can be administered to a stem cell on a substrate including bone-related extracellular matrix proteins. Examples of bone-related extracellular matrix proteins include, without limitation, collagens, vitronectins, fibronectins, tropoelastin, and gelatin.

Effective doses of a composition containing an epigenetic drug, an actin modulator, and one or more osteogenic differentiation factors can vary depending on the route of administration, the age and general health condition of the subject, excipient usage, the possibility of co-usage with other therapeutic treatments such as use of other agents, and the judgment of the treating physician.

An effective amount of a composition containing an epigenetic drug, an actin modulator, and one or more osteogenic differentiation factors can be any amount that enhances bone cell differentiation without producing significant toxicity to the mammal. The effective amount can remain constant or can be adjusted as a sliding scale or variable dose depending on the mammal's response to the composition. Various factors can influence the actual effective amount used for a particular application. For example, the frequency of administration, duration of treatment, use of multiple treatment agents, and route of administration may require an increase or decrease in the actual effective amount administered.

The frequency of administration can be any frequency that enhances bone cell differentiation without producing significant toxicity to the mammal. For example, the frequency of administration can be from about once a week to about three times a day, from about twice a month to about six times a day, or from about twice a week to about once a day. The frequency of administration can remain constant or can be variable during the duration of treatment. A course of treatment with a composition containing an epigenetic drug, an actin modulator, and one or more osteogenic differentiation factors can include rest periods. For example, a composition containing an epigenetic drug, an actin modulator, and one or more osteogenic differentiation factors can be administered daily over a two week period followed by a two week rest period, and such a regimen can be repeated multiple times. As with the effective amount, various factors can influence the actual frequency of administration used for a particular application. For example, the effective amount, duration of treatment, use of multiple treatment agents, and route of administration may require an increase or decrease in administration frequency.

An effective duration for administering a composition containing an epigenetic drug, an actin modulator, and one or more osteogenic differentiation factors can be any duration that enhances bone cell differentiation without producing significant toxicity to the mammal. For example, the effective duration can vary from several days to several weeks, months, or years. Multiple factors can influence the actual effective duration used for a particular treatment. For example, an effective duration can vary with the frequency of administration, effective amount, use of multiple treatment agents, and route of administration.

The invention will be further described in the following examples, which do not limit the scope of the invention described in the claims.

### EXAMPLES

### Example 1—Modulation of the Histone H3K27 Methyltransferase EZH2 Stimulates WNT, PTH and BMP2-Related Paracrine Signaling to Promote Osteogenesis

Bone stimulatory therapeutics that promote bone formation include bone morphogenetic proteins (e.g., BMP2) and intermittent treatment with parathyroid hormone (PTH) or PTH related protein, as well as antibody-suppression of WNT inhibitors (e.g., SOST). Furthermore, inactivation of EZH2, an epigenetic regulator with histone 3 lysine 27 (H3K27) methyltransferase activity, using a pharmacological inhibitor (GSK126) is bone anabolic in skeletally mature mice and osteo-protective in estrogen-depleted (ovariectomized) mice. These biological effects are directly related to the ability of EZH2 inhibition to promote osteogenic differentiation and inhibit adipogenic differentiation of mesenchymal stem cells.

The molecular mechanisms by which EZH2 inhibition promotes osteogenic differentiation were assessed. Results from mRNAseq and ChIP-seq analyses suggested that EZH2 inhibition is anti-proliferative and generates a quiescent cellular state by upregulating the CDK inhibitory protein CDKN2A/p16 and downregulating expression of genes required for mitosis. This quiescent state is conducive for expression of bone-related extracellular matrix proteins (e.g., collagens) that support matrix mineralization. It was found that EZH2 inhibition modulates WNT, PTH and BMP signaling. Several Wnt ligands (e.g., Wnt10b, Wnt10a, and Wnt6) are robustly expressed in differentiating MC3T3 cells. Interestingly, the pro-osteogenic Wnt10b was greatly up-regulated by EZH2 inhibition. Similarly, the PTH receptor (Pthr1h) as also enhanced by GSK126 in preosteoblasts.

Western blotting analysis demonstrated that EZH2 inhibition enhanced Smad1/5 phosphorylation, a well-established biomarker for the activation of BMP2 signaling, in MC3T3 cells. Furthermore, EZH2 inhibition stimulated the expression of BMP2-responsive genes, including several genes involved in osteoblast differentiation (e.g., PTH1R, DLX5, SP7, and IBSP).

These results demonstrated that EZH2 controlled paracrine signaling in osteoblasts involving the WNT, PTH and BMP2 pathways to stimulate osteogenic differentiation, and suggested that inhibitors of EZH2, which include well-tolerated and orally available drugs, may be effective in stimulating bone acquisition by supporting the endogenous local activation of natural bone stimulatory ligands at physiological doses in bone.

### Example 2—Cytochalasin D Improves the Osteogenic Potential of Human Adipose-Derived Mesenchymal Stem Cells Concomitant with Repression of EZH2 and Heterochromatin-Related H3K27me3 Marks

The effect of CytoD on adipose tissue-derived MSCs (AMSCs) was investigated. AMSCs offer several advantages over other sources of MSCs, particularly in the ease of tissue harvest, isolation and expansion to generate sufficient cell numbers for therapy. Because AMSCs have limited osteogenic potential, it is necessary to design molecular strategies to improve their ability to attain a mature osteoblastic phenotype. The depolymerization of the actin cytoskeleton with CytoD had marked effects to enhance osteogenic differentiation of AMSCs throughout the cell culture time-course, as reflected by significant increases in alkaline phosphatase activity and mineralization, as well as the expression of osteogenic polypeptides RUNX2, ALP, OPG and TGFβ3. RNA-seq analyses of both AMSCs and BMSCs in response to CytoD (24 hour) revealed significant upregulation of a program of other osteogenic markers, including those linked to the BMP2-RUNX2 axis (e.g., SP7). Furthermore, CytoD decreased protein levels of Enhancer of Zeste Homolog 2 (EZH2), an epigenetic suppressor of osteogenic differentiation that mediates heterochromatinization of bone-related genes by trimethylation of histone 3 lysine 27 (H3K27me3). This loss of EZH2 protein is reflected by decreased levels of H3K27me3 marks indicating a global reduction in heterochromatin.

These results demonstrated that actin polymerization is linked to epigenetic mechanisms that control the acquisition of the osteogenic phenotype in AMSCs, and suggested that CytoD advanced the osteogenic potential of AMSCs facilitating their use in skeletal regenerative strategies.

### Other Embodiments

It is to be understood that while the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims.

1. A composition comprising an epigenetic drug, an actin modulator, and one or more osteogenic differentiation factors.

2. The composition of claim 1, wherein the epigenetic drug inhibits enhancer of zeste homolog 2 (EZH2) polypeptide activity.

3. The composition of claim 2, wherein the epigenetic drug is GSK126.

4. The composition of claim 1, wherein the actin modulator inhibits actin polymerization.

5. The composition of claim 4, wherein the actin modulator is cytochalasin D (CytoD).

6. The composition of claim 1, wherein the one or more osteogenic differentiation factors are pro-osteogenic differentiation factors selected from the group consisting of Wnt10b, a sclerostin antibody, BMP2, BMP5, teriparatide, and abaloparatide.

7. (canceled)

8. The composition of claim 1, wherein the epigenetic drug is GSK126, wherein the actin modulator is CytoD, and wherein the one or more osteogenic differentiation factors are selected from the group consisting of Wnt10b, a sclerostin antibody, BMP2, BMP5, teriparatide, and abaloparatide.

9. (canceled)

10. A method for enhancing bone cell differentiation, the method comprising administering to a stem cell a composition comprising an epigenetic drug, an actin modulator, and one or more osteogenic differentiation factors, wherein the stem cell is differentiated into a bone cell.

11. The method of claim 10, wherein the stem cell is an adult stem cell selected from the group consisting of a mesenchymal stem cell, an adipose-derived stem cell, and a bone marrow-derived stem cell.

12. (canceled)

13. The method of claim 11, wherein the adult stem cell is an adipose-derived stem cell.

14. The method of claim 10, wherein the bone cell is an osteocyte.

15. The method of claim 10, wherein the stem cell is from a mammal.

16. The method of claim 15, wherein the mammal is a human.

17. The method of claim 10, wherein the epigenetic drug is GSK126.

18. The method of claim 10, wherein the actin modulator is cytochalasin D (CytoD).

19. The method of claim 10, wherein the one or more osteogenic differentiation factors are selected from the group consisting of Wnt10b, a sclerostin antibody, BMP2, BMP5, teriparatide, and abaloparatide.

20. The method of claim 10, wherein the stem cell is in vivo.

21. The method of claim 10, wherein the stem cell is in vitro.

22. The method of claim 21, wherein the stem cell is on a substrate.

23. The method of claim 22, wherein the substrate comprises a bone-related extracellular matrix protein.

24. The method of claim 23, wherein the bone-related extracellular matrix protein is a collagen.

25. A method for increasing polypeptide expression in a stem cell, the method comprising contacting the stem cell with a composition comprising an epigenetic drug, an actin modulator, or one or more osteogenic differentiation factors, wherein expression of one or more osteogenic polypeptides is increased.

26.-36. (canceled)
